(19) United States
(12) Patent Application Publication
(10) Pub. No.: US 2025/0281204 A1
(43) Pub. Date: Sep. 11, 2025

(54) STABILIZING AND FUSING JOINTS

(71) Applicant: Lenoss Medical, Inc., Bristol, RI (US)

(72) Inventors: Dom Messerli, Bristol, RI (US); Kevin Cooney, Bristol, RI (US)

(21) Appl. No.: 19/054,386

(22) Filed: Feb. 14, 2025

Related U.S. Application Data
(60) Provisional application No. 63/553,996, filed on Feb. 15, 2024.

(51) Int. Cl. A61B 17/56

(52) U.S. Cl. CPC A61B 17/56; A61B 2017/564

(57) ABSTRACT

An approach to stabilizing the sacroiliac (SI) joint employs flexible, structurally supportive implants that adapt to the complex anatomy of the SI joint. The associated methods are less invasive and are able to navigate the complex structure of the SI joint and its surrounding muscles, ligaments, and neural network. The implants can follow the joints' natural anatomy, fill the joint space, and create an extensive fusion base to provide stability to the SI joint.

### RELATED APPLICATIONS

This application claims priority to U.S. Provisional Patent Application No. 63/553,996, filed Feb. 15, 2024, the entirety of which is incorporated by reference herein.

### TECHNICAL FIELD

This specification generally relates to stabilizing and fusing joints, such as the Sacroiliac (SI) joint.

### BACKGROUND

Joints are part of your skeletal system where two bones meet. Joints are often classified based on how they move and what they are made of. Synarthroses joints are joints that don't move at all but rather provide structural support. Amphiarthroses joints are joints with limited movement that provide a mix of stability and motion. Diarthroses joints are joints can move freely in most directions.

The SI joint is a diarthrodial joint located in the pelvis that connects the sacrum (the triangular bone at the base of the spine-with a series of ridges and depressions on its auricular surface (ear-shaped surface)) to the ilium (the large, wing-shaped bone of the pelvis). The joint surfaces are irregular and have ridges and depressions that interlock, forming a stable yet slightly movable joint. Although the range of motion at the sacroiliac joint is limited compared to other joints in the body, it does allow for a small amount of movement, primarily in the form of nutation (forward tilting) and counternutation (backward tilting) of the sacrum relative to the ilium.

The SI joint is also a synovial joint, meaning it has a joint capsule and contains synovial fluid to lubricate the joint surfaces. The sacroiliac joint is surrounded by a fibrous joint capsule, which is relatively thin anteriorly and posteriorly but thicker laterally. The capsule helps provide stability to the joint and contains synovial fluid to reduce friction and facilitate smooth movement.

The SI joint is a complex structure. It is surrounded by numerous nerves, ligaments, and muscles, all of which play critical roles in stabilizing and supporting the joint and preventing excessive movement. These ligaments include the anterior and posterior sacroiliac ligaments, interosseous ligament, and the sacrotuberous and sacrospinous ligaments.

There are also a series of muscles and nerves that are part of this complex anatomy.

The SI joint plays an important role in transferring weight and forces between the upper body and the legs. The primary functions of the sacroiliac joint include transmitting forces from the upper body to the pelvis and lower extremities, providing stability and support for the spine, and accommodating movement of the pelvis during activities such as walking, running, standing, and bending. Its function is crucial for overall mobility and stability. Due to its intricate anatomy and its role in weight-bearing and movement, issues with the SI joint can lead to significant discomfort and dysfunction for individuals.

The SI joint can be prone to various problems and dysfunctions, which can lead to pain and discomfort in the lower back, buttocks, hips, and groin area. Common conditions affecting the sacroiliac joint include sacroiliac joint dysfunction, sacroiliitis (inflammation of the joint), and sacroiliac joint arthritis. Sacroiliac Joint Dysfunction (SIJD) occurs when there is abnormal movement or alignment of the sacroiliac joint. It can result from trauma, pregnancy-related changes, repetitive stress, or degenerative changes. SIJD can cause pain and stiffness in the lower back, buttocks, and hips, often exacerbated by certain activities such as standing, walking, or climbing stairs. Sacroiliitis refers to inflammation of the sacroiliac joint. It can be caused by various factors, including infection, autoimmune diseases such as ankylosing spondylitis, or mechanical stress. Sacroiliitis typically presents with pain and tenderness in the lower back and buttocks, often worsening with prolonged sitting or lying down.

Fractures of the sacrum, the triangular bone at the base of the spine that forms part of the sacroiliac joint, can occur due to trauma, such as falls or motor vehicle accidents. Traumatic injuries, such as a direct blow to the pelvis or a fall onto the buttocks, can cause damage to the sacroiliac joint, resulting in pain, swelling, and bruising in the lower back and pelvic region. Sacral fractures can cause severe pain in the lower back and pelvic region, along with difficulty walking or standing.

The sacroiliac joint can also undergo degenerative changes, similar to other weight-bearing joints in the body. Osteoarthritis of the SI joint can lead to cartilage breakdown, bone spurs, and joint stiffness, resulting in chronic pain and reduced mobility. During pregnancy, hormonal changes can affect the ligaments and stability of the pelvis, leading to increased stress on the sacroiliac joint. This can result in pregnancy-related SI joint pain, characterized by sharp or dull pain in the lower back and buttocks, especially with activities like walking or standing for prolonged periods.

Mechanical strain and injury to the SI joint are produced by either a combination of vertical compression and rapid rotation (i.e. carrying a heavy object and twisting), or by falls on the backside. Injuries of this type can produce ligamentous laxity and allow painful abnormal motion, in other words, SI joint pain. Instability of the sacroiliac joint can also arise from lumbar spine surgery in which a large portion of the ilio-lumbar ligament is injured. SI joint pain can also be caused by leg length discrepancy, gait abnormalities, prolonged, vigorous exercise, trauma, traumatic birth, and long scoliosis fusions to the sacrum. Painful sacroiliac joint arthritis can also arise from autoimmune disorders, such as ankylosing spondylitis, juvenile rheumatoid arthritis, Reiter's Syndrome, psoriatic arthritis, and infections including staphylococcus, gonorrhea and TB.

### SUMMARY

This specification describes an approach to stabilizing and fusing joints, such as the SI joint. This approach provides a low impact alternative to interventions such as surgical SI joint fusion may be necessary to treat for sacroiliac joint problems when conservative measures such as physical therapy, activity modification, pain medications, bracing, or pain injections (e.g., corticosteroid injections) are unsuccessful. This approach uses a flexible chain of implants that include body portions joined by flexible links (also referred to as struts). These flexible implants can adapt to the structural anatomy of, for example, the SI joint rather than requiring creation of a bone void to place the implants. In addition, these implants can be placed through a cannula as small as an 11 gauge access cannula (OD appx 4 mm and ID appx 3.8 mm) rather than requiring the larger size instruments (e.g., drills, dilation tools, large square or rectangular shaped tools, reamers, rasps, and broaches) required by some procedures. This approach is well-suited to the complex anatomy of the SI joint as the small access point easily navigates the SI joint's surrounding muscles, ligaments, and neural network and does not require extensive bone removal as the cannula may be inserted directly into the SI joint.

SI joint fusion is a surgical procedure performed to stabilize the joint by fusing the sacrum and the ilium together. But the complex anatomy of the SI joint with its complicated array of bones, muscles, ligaments, and nerves requires careful consideration and expertise from surgeons performing surgical procedures targeting this area making precise localization crucial to avoid damage to surrounding structures. Surgeons must consider the various muscular attachments during surgery to minimize disruption and facilitate optimal postoperative recovery. The complex neural network of the SI joint requires surgeons to exercise extreme caution to avoid damaging these nerves because there can be serious postoperative complications such as pain or sensory deficits. There can also be variability in the anatomy of the SI joint among individuals, including differences in the size, shape, and orientation of the joint which further complicates the procedure. Surgeons must be prepared to adapt their approach based on these variations to ensure optimal outcomes.

The approach described in this specification can provide one or more of the following advantages.

This approach avoids intricate navigation of the SI joint anatomy and long recovery time associated SI joint fusion performed using an open procedure where the joint is exposed so the surgeon can see the iliac and the sacral joint surfaces. In open procedures, once the joint surfaces are exposed the articular cartilage is removed from the surfaces of the iliac and sacrum, then the bones are held together with metal plates or screws across the joint for the bones to fuse together.

This approach is also less complex than and has a reduced likelihood collateral damage relative to minimally invasive (MIS) techniques based on making small incisions using special instruments to access the SI joint. During these kinds of procedures, hardware such as plates, screws, or rods may be used to stabilize the SI joint; and in certain instances, bone graft material is placed between the sacrum and ilium to promote fusion of the joint.

For example, one MIS approach requires an incision, about one to two inches long, along the side of the buttock. Instruments are then used to remove bone to create access through the bone to the joint and to prepare the bone for placement of the implants. The surgeon, for example, uses special instruments to guide the drill that creates access to the SI joint and provides the opening through which the implants are inserted. These fusion surgical procedures also generally use rasps and/or broaches to prepare the surfaces of bones, sometimes to cause bleeding to provide a better surface and environment for fusion. As is common to all surgical techniques, the surgeon does this procedure under fluoroscopy to allow for real-time images of the SI joint anatomy during the procedure. The surgeon typically places three implants across the SI joint to fuse the joint together through three separate access points. Other MIS approaches also use various screws and hardware that require drilling and rasping is used to remove bone.

This approach can also reduce the likelihood of disrupting and/or injuring the intraosseus ligament associated with implants that are placed using a posterior approach. The intraosseous ligament helps to stabilize the SI joint, which is critical for maintaining proper alignment and function of the pelvis and lower back. In particular, the intraosseous ligament plays a role in transferring loads and forces between the sacrum and ilium during weight-bearing activities. Disrupting its function can disrupt normal biomechanical function, potentially leading to changes in gait, posture, and movement patterns, which may contribute to pain and dysfunction in the lower back and pelvis. Injury to this ligament during surgery can result in postoperative pain and discomfort around the SI joint region. Injury to the intraosseous ligament during SI joint surgery can increase the risk of complications such as infection, hematoma formation, nerve injury, and vascular damage. These complications can prolong recovery, increase patient discomfort, and potentially necessitate additional interventions to manage, including additional pain management strategies. The desired surgical outcome for an SI joint fusion is to stabilize the joint, and damage to this ligament can impair the healing process and compromise that outcome and may even lead to revision surgery or alternate treatment.

The described implants and methods do not require a surgeon to remove bone to create space for the implants to fit in, but rather, these implants can adapt to the SI joint anatomy. Because these implants are flexible and also structural, they allow for a less invasive surgical and bone preserving procedure because large amounts of bone do not need to be removed via drills, or aggressive rasping and reaming to fit the implants. In general, this approach does not require bone removal because the access cannula is placed directly into the joint from, for example, the posterior-caudal aspect of the joint that provides direct access into the joint without any bone removal. If any bones needs to be removed, it is only as much bone as needed to make space for a cannula to create access to the joint and for implant deployment. There is some reaming that occurs as in any fusion procedure to prepare the joint surface, but the reaming process is less traumatic because of the adaptable nature of the implant. This all adds up to a less invasive, bone preserving procedure, less operating room time, less exposure to fluoroscopy, and generally a shorter recovery time.

In contrast to the structural implants of other approaches where a bone void needs to be created to place the implants, the flexible implants of this approach adapt to the structural anatomy of the SI joint and may be placed through a cannula as small as an 11 gauge access cannula (OD appx 3 mm and ID appx 2.4 mm), as opposed to the larger size instruments that are required for the procedures discussed above. For example, these other procedures often require multiple access instruments, such as drills; dilation tools; large square or rectangular shaped tools; reamers; rasps; and broaches to create the space in the anatomy that is needed to accommodate the particular device or devices that need to be implanted. This approach is well-suited to the complex anatomy of the SI joint as the small access point easily navigates the SI joint's surrounding muscles, ligaments, and neural network and do not require extensive bone removal as the cannula may be inserted directly into the SI joint

The details of one or more embodiments of these systems and methods are set forth in the accompanying drawings and the description below. Other features, objects, and advantages of these systems and methods will be apparent from the description and drawings, and from the claims.

### DESCRIPTION OF DRAWINGS

FIGS. 1A and 1B illustrate an approach to fusing an SI joint without bone removal. FIG. 1A is a 3D CT scan of an SI joint. FIG. 1B is a 2D CT scan of the SI joint of a cadaver after insertion of implants using the described approach.

FIG. 2 is a flowchart of a method of stabilizing and/or fusing an SI joint.

FIGS. 3A-3D illustrate the ability of the approach to avoid damage or penetration to the dorsal intraosseous ligaments. FIG. 3A is a schematic representation illustrating the location of a dorsal interosseous ligament and a ventral articular cartilage portion of the SI joint. FIG. 3B is a picture of an SI joint showing the highly irregular shape of the joint. FIG. 3C is a 3D radiographic image with dorsal interosseous ligament outlined. FIG. 3D is a radiographic image of a trial of the approach.

FIG. 4A is a perspective view of a chain implant. FIG. 4B is a close up cross-sectional view of chain, taken at line B-B in FIG. 4A.

FIGS. 5A-5C are perspective views of other implants 200 with other geometric configurations.

FIG. 6 is a schematic illustration of a flexible implants formed from a demineralized allograft.

FIGS. 7A-7D are examples of implants formed by 3D printing methods and systems rather than by milling, shaping, demineralizing, and dehydrating a cortical bone.

FIGS. 8, 9A, and 9B illustrate how the implant adapts to the natural curvature and anatomy of the joint. FIG. 8 is a schematic illustrating how an implant inserted in a straight line adapts and fills the joint. FIGS. 9A and 9B are radiographic images with, in FIG. 9B, an image of the implant imposed over the radiographic image.

FIG. 10A illustrates using a dual cannula (i.e., a cannula with two access tubes) to deliver implants. FIG. 10B illustrates using a triple cannula (i.e., a cannula with three access tubes) to deliver implants.

FIG. 11 illustrates the use of multiple single tube cannulas.

FIG. 12 illustrates a posterior-cranial saddle access approach.

Like reference symbols in the various drawings indicate like elements.

### DETAILED DESCRIPTION

This specification describes an approach to stabilizing and fusing joints such as the SI joint. This approach uses a flexible chain of implants that include body portions joined by flexible links (also referred to as struts). The body portions can be solid, semi-solid or hollow, substantially non-flexible body portions (also referred to as bodies or beads) and a series of flexible link portions. These flexible implants can adapt to the structural anatomy of, for example, the SI joint rather than requiring creation of a bone void to place the implants. In addition, these implants can be placed through a cannula as small as an 11 gauge access cannula (OD appx 3 mm and ID appx 2.4 mm) rather than requiring the larger size instruments (e.g., drills, dilation tools, large square or rectangular shaped tools, reamers, rasps, and broaches) required by some procedures. This approach is well-suited to the complex anatomy of the SI joint as the small access point easily navigates the SI joint's surrounding muscles, ligaments, and neural network and does not require extensive bone removal as the cannula may be inserted directly into the SI joint.

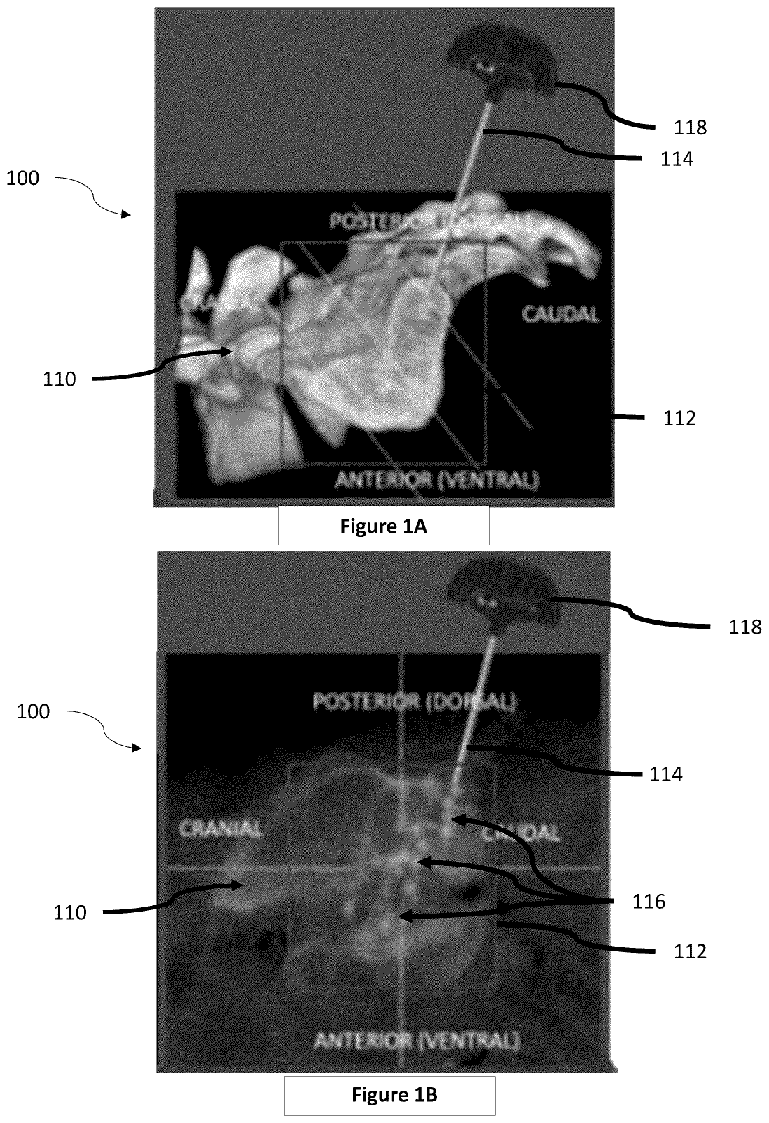

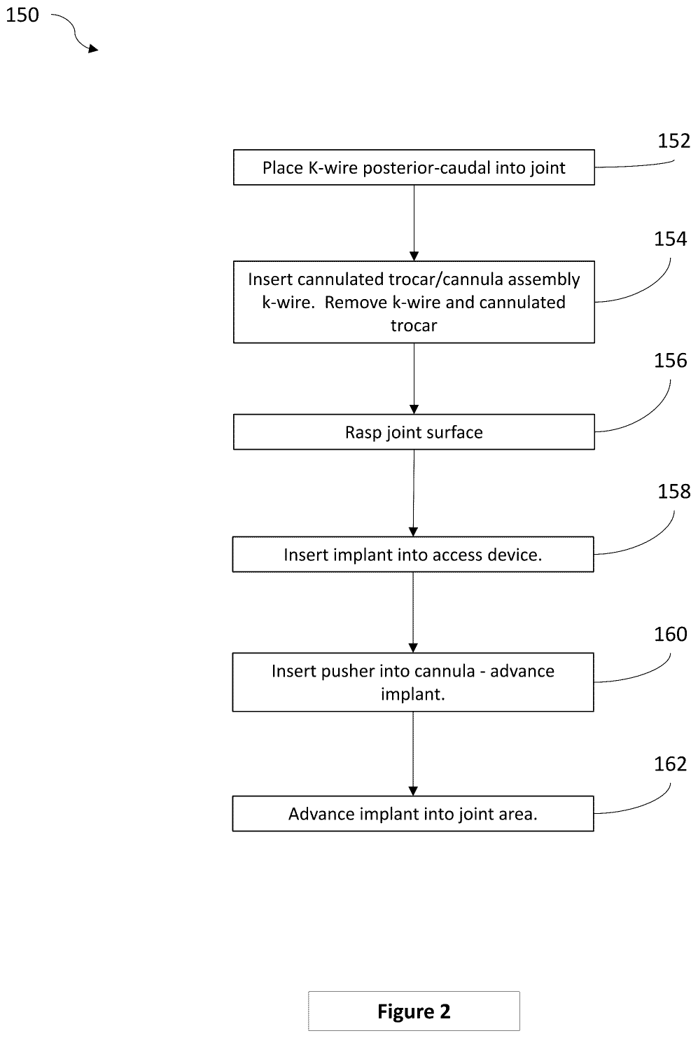

FIGS. 1A and 1B illustrate an approach 100 to fusing an SI joint 110 without bone removal. FIG. 1A is a 3D CT scan of an SIjoint. FIG. 1B is a 2D CT scan of the SI joint 110 of a cadaver after insertion of implants using the described approach. FIG. 2 is a flowchart of a method 150 of stabilizing and/or fusing an SI joint. The method 150 can be applied using at various entry points to the joint and via various numbers of entry sites.

The approach 100 can be characterized as a single minimally invasive surgery (MIS) posterior-caudal approach. The box indicates the articular area 112 of the SI joint 110. An access device is used to insert implants into the articular area 112 of the SI joint 110. As illustrated, a cannula 114 is used to insert a flexible chain of implants 116 but other access devices such as square shaped, octagon shaped or curved cannulas can also be used to insert the flexible chain of implants 116.

A Kirschner wire (K-wire—not shown) is placed posterior-caudal into the SI joint 110 (step 152). K-wires are a thin, straight, and sharpened metal wire used to temporarily stabilize bones during procedures and/or healing. A cannulated trocar/cannula assembly is inserted into the SI joint 110 along the k-wire (step 154). The k-wire and the cannulated trocar are removed leaving a cannula 114 placed in the SI joint 110. In some cases, a tool is used to slightly rasp of the joint surface to create bleeding bone (step 156). The flexible chain 116 of implants are inserted into the cannula 114 (step 158). A pusher 118 is inserted into cannula 114 (step 160) advances the chain of implants 116 out of the cannula 114 (step 162) into the articular area 112 of the SI joint 110 as illustrated in FIG. 1B. The implant's multi planar flexibility of the implant 116 allows it to fill the SI joint space and adapt to the irregularly shaped anatomy. The access point for this technique is much smaller that those required by techniques using drilling and larger access instruments to accommodate other implants.

The small implant size allows the use of the small, minimally invasive access cannula and therefore allows the end user physician to place the access cannula in the exact same manner as he would for a standard pain injection into the SI-Joint. Despite using the minimally invasive “pain-injection” approach, the implant will still be able to disburse into the joint space and fully fill the joint area for optimal stability and fusion.

The radiographic imaging methods to place the access cannula into the proper anatomical location to enter the joint space are the same as the ones being used for the standard pain injections into the joint. No additional images are required for assuring accuracy and safety of the approach, versus other current methods may require additional view and imaging to assure the large approach instruments are not penetrating or damaging vital anatomical structures.

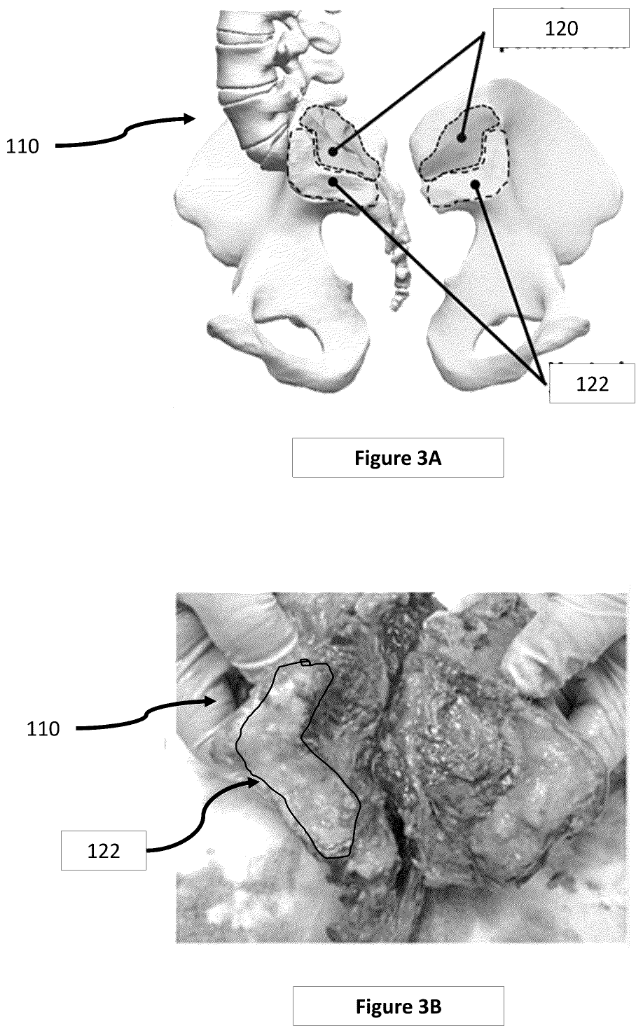

FIGS. 3A-3D illustrate the ability of the approach 100 to avoid damage or penetration to the dorsal intraosseous ligaments. FIG. 3A is a schematic representation illustrating the location of a dorsal interosseous ligament 120 and a ventral articular cartilage portion 122 of the SI joint 110. FIG. 3B is a picture of an SI joint 110 showing the highly irregular shape of the joint. This is a view of the joint pulled apart like opening a sandwich. In this image one can see the multi planar, generally L-Shaped irregular shape of the joint.

FIG. 3C is a 3D radiographic image with dorsal interosseous ligament 120 outlined. FIG. 3D is a radiographic image of a trial of the approach 100. A cannula 114 inserted into the SI joint 110 is visible in both FIG. 3C and FIG. 3D with the chain of implants 116 visible in FIG. 3D. By avoiding dorsal intraosseous ligament 120, the approach 100 can preserve the function of the ligament helping maintain joint stability. In contrast, many available implant system designs that force the dorsal approach disrupt the interosscous ligament.

The multi-directional flexibility and adaptability of the implant can allow for the implant to follow the irregular, multiplanar shapes of the sacroiliac joint and fill the entire joint. In addition, the ability to deliver a varying amount of implant that can fill a majority of the irregular, multiplanar shaped sacroiliac joint facilitates a wider fusion bed and joint stability. Furthermore, the post implantation implant volume increase due to the implants ability to hydrate with the patients' blood, increased void fill and pressurization of the void allows for increased stability and therefore optimized fusion and pain relieve.

The implants may be made biocompatible material having, for example, the desired characteristics of flexibility, strength, and other desired properties that enable the implants to adapt to the joint anatomy. Some of the materials that can be used for the implants are bone (e.g., cortical, cancellous bone, or both); polymers (e.g., PEEK, PEKK, or material from the PAEK family); metals (e.g., stainless steel, titanium, nitinol, cobalt chrome); ceramics; xenografts; autografts; allografts; composites. Some implants are made of combinations of two or more of these materials. The bodies and the linking parts of the implants may be formed from the same or different material, they may be the same or different sizes, they may be uniform or nonuniform, or a combination of any of the foregoing.

For example, the implants can have a demineralized layer which can increase new bone formation through enhanced osteoconduction and osteoinduction, which may be rendered osteogenic once mixed with the patient's blood during the procedure. The monolithic strand can include or be formed by cortical bone which can increase stability and compressive strength. Some assemblies may include an implant device with a monolithic elongated body formed by either a unitary strand or formed by a chain of interconnected links. The elongated body includes large diameter sections, described as beads and/or sockets, connected to each other by small diameter sections, described as joints and/or shaped bodies. The joints are flexible or rotatable so that the elongated body is collapsible and folds when compressed. The implant can then be manipulated as needed to form a thin, elongated string or to form a dense cluster or mass. In the folded configuration, the elongated body folds at the joints to move the beads towards each other. The disclosed implants and methods provide a natural, flexible strand of material that is generally provided in a dehydrated state but can then be rehydrated prior to the surgical procedure. In the folded configuration within the joint, the 3 dimensional macro structure allows for ample blood and nutrient in- and through flow to allow for enhanced bony fusion.

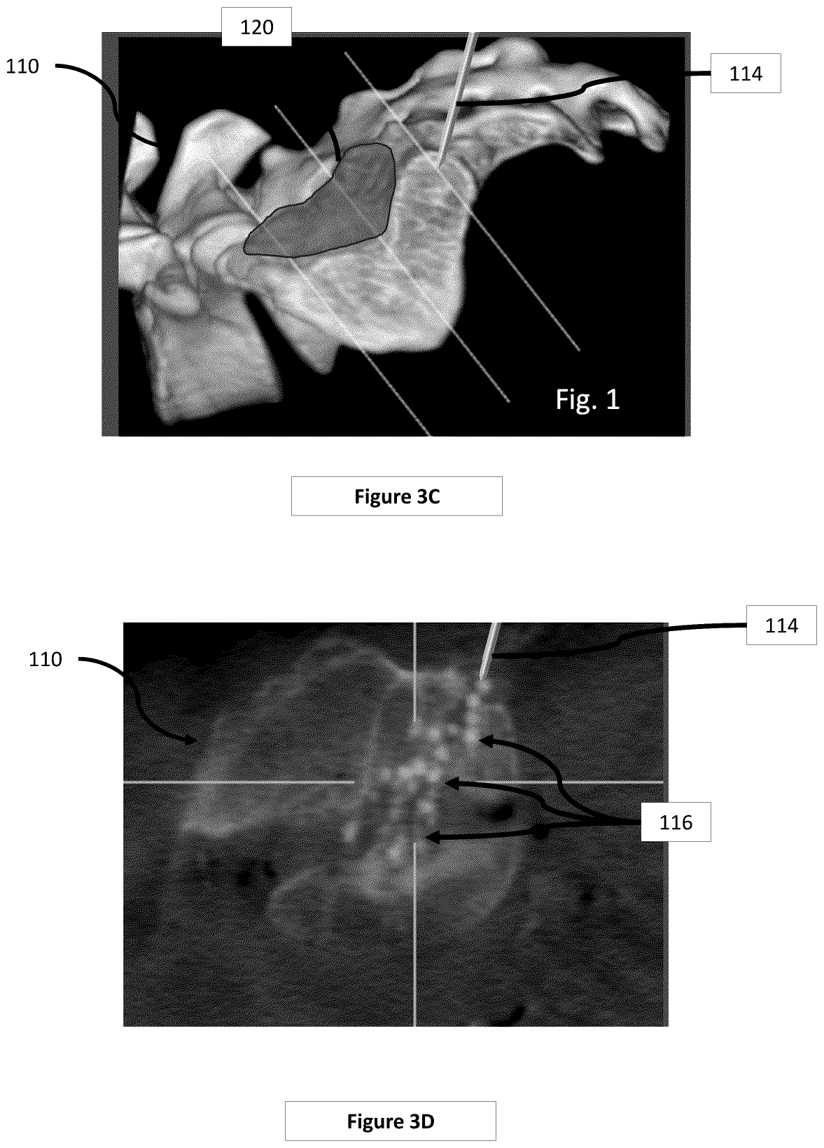

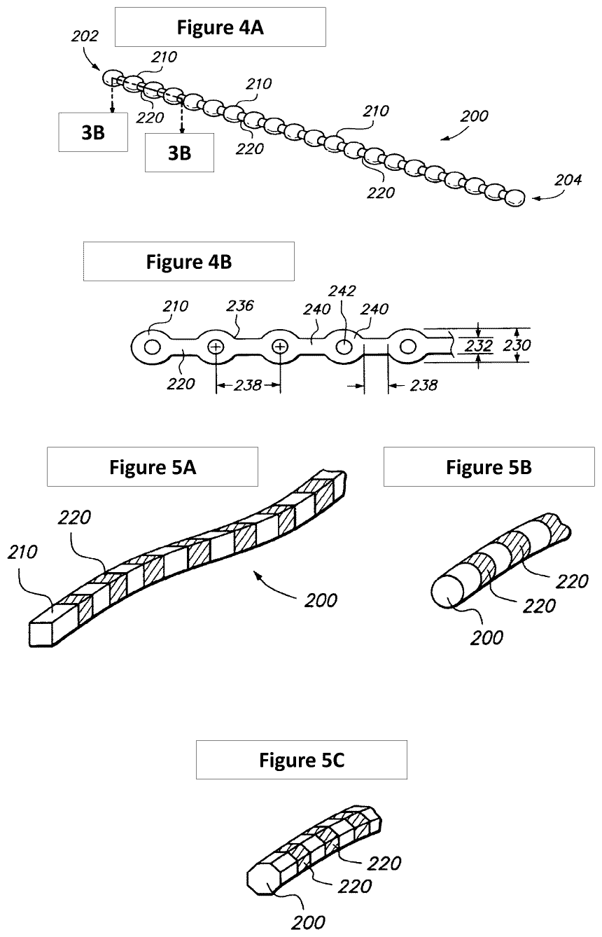

FIG. 4A is a perspective view of an implant 200. The implant or chain 200 includes one or more bodies 210 (sometimes referred to as beads). Chain 200 can be a monolithic chain, e.g., formed from a single, common material or type of material forming an integral structure. Bodies 210 are preferably substantially non-flexible, and may be solid, semi-solid, porous, non-porous, hollow, or any combination thereof. Chain 200 may also comprise one or more linking portions 220, also sometimes referred to as struts or links 220. Struts 220 may be disposed between each pair of adjacent bodies 210. Struts 220 are preferably substantially flexible or semiflexible, e.g. to allow for bending of the chain 200 between bodies 210.

The bodies 210 may be absorbable or resorbable by the body. For some applications, the bodies 210 preferably have osteoinductive properties or are made at least partly from osteoinductive materials. The outer circumferential shape of the body may be the same as adjacent links. Alternatively or in addition, the outer circumferential shape of the body may be the same size as adjacent links. Bodies 210 may be of uniform or non-uniform size, shape and/or materials, and may be linked in series, for example by one or more flexible or semi-flexible linking portions 220, which can form struts of any desired length between bodies 210. Linking portions are preferably, although not necessarily, formed of the same material as bodies 210.

FIG. 4B is a close up cross-sectional view of chain 200, taken at line B-B in FIG. 4A. In this example, chain 200 is a monolithic chain, with bodies 210 and flexible portions 220 formed from a uniform material, e.g., bone. Although bodies 210 are shown as substantially spherical, and linking portions 220 are shown as substantially cylindrical, numerous other shapes are contemplated. In fact, chains 200, including body 210 and/or linking portion 220, may be of any desired shape, such as for example, cylindrical, elliptical, spherical, rectangular, etc. Body 210 and/or linking portion 220 may also be of any particular cross sectional shape such as round hexagonal, square, etc. Bodies 210 and linking portions 220 may have the same or different shapes. In certain embodiments the configurations of bodies 210 may vary within a chain 200. Alternatively or in addition thereto, the configuration of links 220 may vary within a chain. In one embodiment, the bodies can be shaped so that they fit together to minimize interstitial spacing or provide a predetermined range of interstitial spacing.

FIGS. 5A-5C are perspective views of other implants 200 with other geometric configurations. For example, rigid portions 210 and flexible portions 220 may have the same or different shapes, such as cubes, cylinders, any polyhedral shapes, balls, banana or kidney shaped, or any combination thereof. Portions 210 and/or 220 may have any desired cross-sectional shape, such as for example rectangular, circular, elliptical, pentagonal, hexagonal, etc. The flexible 220 and non-flexible 210 portions may be of the same shape to form relatively uniform shaped structures as shown in FIGS. 5A-C.

Further details of implants usable with the techniques described in this specification are described in U.S. Pat. Nos. 11,406,508 and 9,289,240, and U.S. Provisional Patent Application Ser. No. 63/530,926, the disclosure of which are herein incorporated by reference in their entirety.

The substantially non-flexible body portions preferably are capable of withstanding loads that are applied in any direction, and the flexible link portions of the implant preferably are disposed between the substantially non-flexible body portions and preferably are flexible in any direction, although they may be flexible in only selected or desired directions. The flexible link and non-flexible body portions may be formed from the same material but can be different materials. The flexibility of these implants allow them to follow the irregular shapes of the sacroiliac joint.

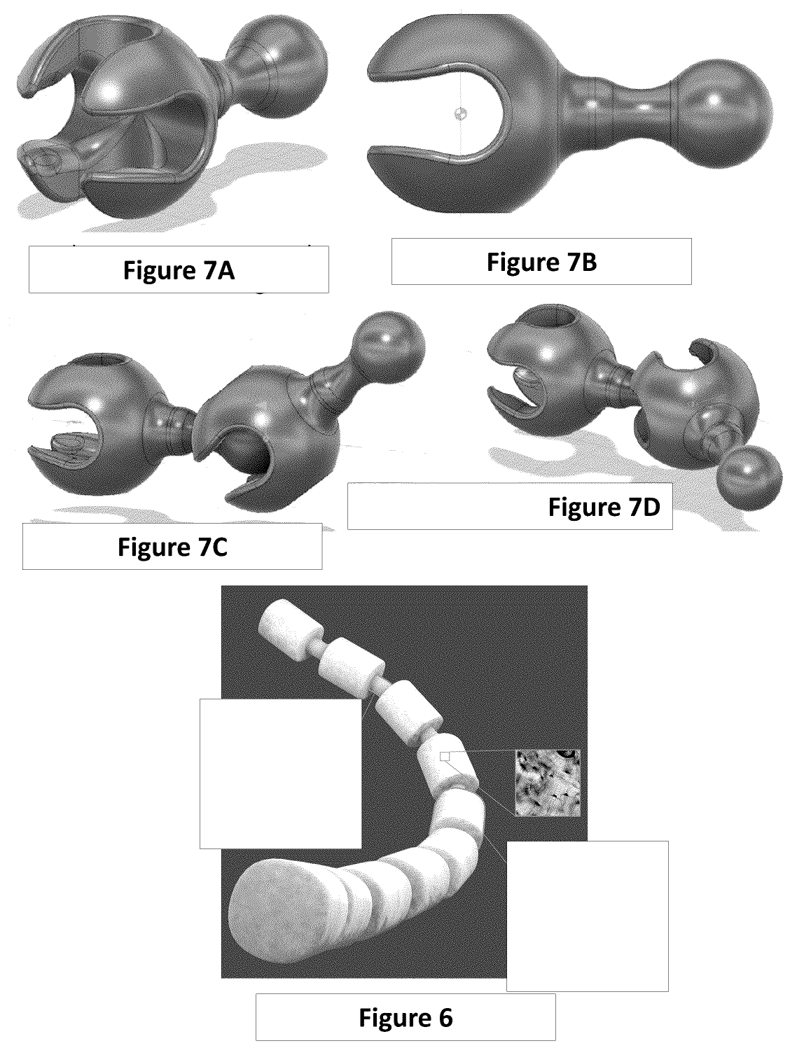

FIG. 6 is a schematic illustration of a flexible implants formed from a demineralized allograft. Such implants can be provided as dehydrated monolithic strands that can be stored at room temperature, for example on a shelf in a hospital. Such implants are prepared with a pre-soaking step before implantation where the implant is inserted into a saline bath for a period of time (e.g., about 3 minutes). The implants increase from a dehydrated volume to a partially rehydrated volume which further increases the flexibility of the implants. The partially hydrated, flexible implants can be inserted into a cavity of a body and is then further swollen, in situ, to a rehydrated volume when mixed with the body fluids including the patient's blood. The rehydrated strand increases in volume, for example, by about 40% from the dehydrated volume to the rehydrated volume and this expansion provides structural stability to the SI joint and provides a larger fusion bed.

The implant bodies may be different sizes and shapes than the links or they may be the same shape, same size, or both. In addition, each body and link may be a different size and shape than other bodies or links. In one embodiment, the beads can be shaped so that they can fit together to minimize interstitial spaces. For example, the beads may be shaped as cubes or other polyhedrals that can be stacked together in such a way that there is little space between beads, or a predetermined percentage range of interstitial space. The elongated member may be formed as an integral monolithic chain, which may be formed of bone, such as, for example, allograft bone. The flexible links may be formed of bone that has been demineralized to a greater extent than the bodies. Optionally, a coating may be applied to at least a portion of the elongated member, e.g. a coating comprising a therapeutic agent, a bone cement, an antibiotic, a bone growth stimulating substance, bone morphogenic protein (BMP) or any combination thereof. Therapeutic agents, or drug agents (e.g., antibodies), or biologics (e.g., one or more BMPs) can be coated, or attached via peptides, adsorbed, absorbed or in some other way perfused onto or into the elongated member; either the bodies, the links or both. In some embodiments, the coating may comprise a bone cement that may be activated upon insertion into the bone. In other embodiments, at least a portion of the bodies comprise an outer surface configured to promote bone in-growth.

These strands of implants can be formed by a variety of procedures including milling, shaping, demineralizing, and dehydrating (e.g., by freeze-drying) and which can be swollen immediately prior to insertion into a body in a pre-soaking step. The monolithic strand can be formed using biological materials, through bone milling, or using inorganic materials. In a monolithic (unitary, single material) strand, the beads and joints are integrally connected and are generally sculpted and shaped from a single piece of material. In some implants, the unitary strands are formed by extruding or printing an elongated strand of joints and bead. The extruded or printed strands are infilled with at least one bone-like infill pattern which can mirror the structure of a cortical bone or a cancellous bone. The bone-like infill pattern can promote bone growth during the healing process.

FIGS. 7A-7D are examples of implants formed by 3D printing methods and systems rather than by milling, shaping, demineralizing, and dehydrating a cortical bone. In 3D printed strands or chains, the implants can include specially printed surfaces (exteriors) and infill structures to increase surface area available for osteointegration and osteoconduction. The infill structures, for example, can imitate or mimic the structure of a cortical bone or cancellous bone to promote osteointegration and osteoconduction. Further, the implants can provide smooth surfaces at locations of the implant that experience high levels of friction. For example, where the implant is formed by connected links which attached to each other by ball-and-socket attachment structures, the sockets can include smooth inner surfaces to decrease wear and material degradation. Additionally, the implant can have customized or adjustable modulus of elasticity between different portions of the implant. For implants that are 3D printed, for example, as a monolithic print of a polymeric material having both beads and links printed as a single chain, the linked chain may be formed by 3-D printing each individual link in connection with each other. It is important to note that the attachment structures are printed within each other. It is not possible to machine this design, it can only be 3D printed as the ball is printed within the socket feature. Therefore, the ball can never escape the socket as it is fully constraint by the socket.

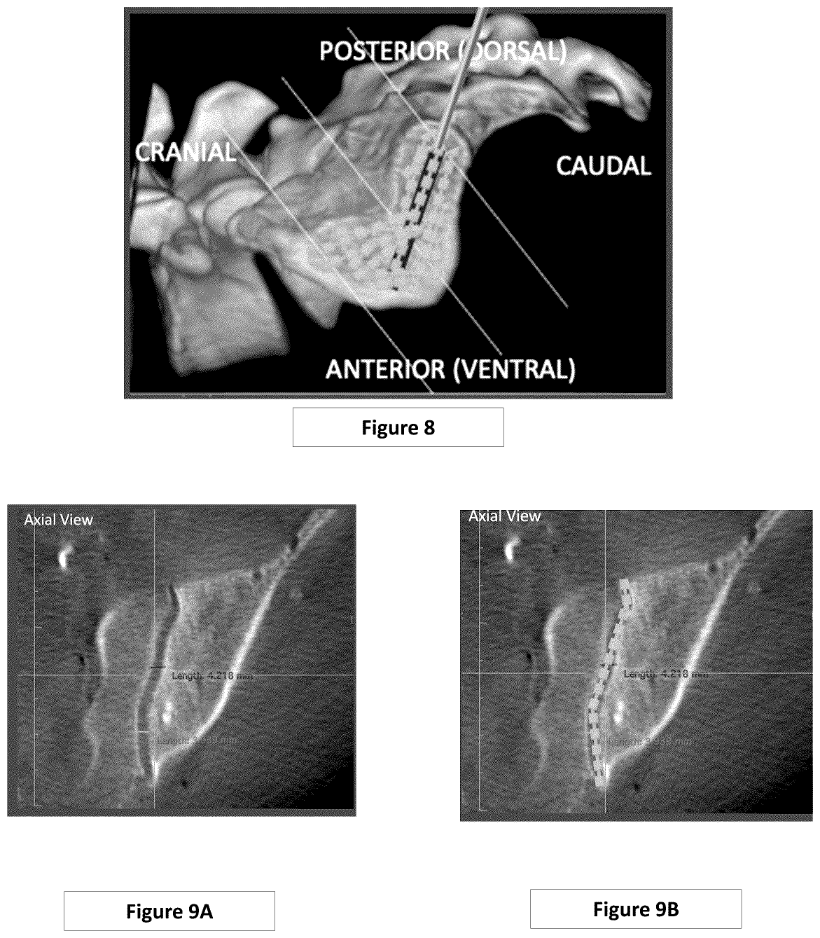

FIGS. 8, 9A, and 9B illustrate how the implant adapts to the natural curvature and anatomy of the joint. FIG. 8 is a schematic illustrating how an implant inserted in a straight line adapts and fills the joint. This distracts the joint, increases the joint stability because of the ability of the implant to fill the joint, and promotes fusion with a larger fusion bed than other structural and solid implants that are on the market. FIGS. 9A and 9B are radiographic images with, in FIG. 9B, an image of the implant imposed over the radiographic image. This adaptability is particularly important with respect to the complex anatomical structure of the SI joint in general, and the additional variability based on a patient's sex and stature.

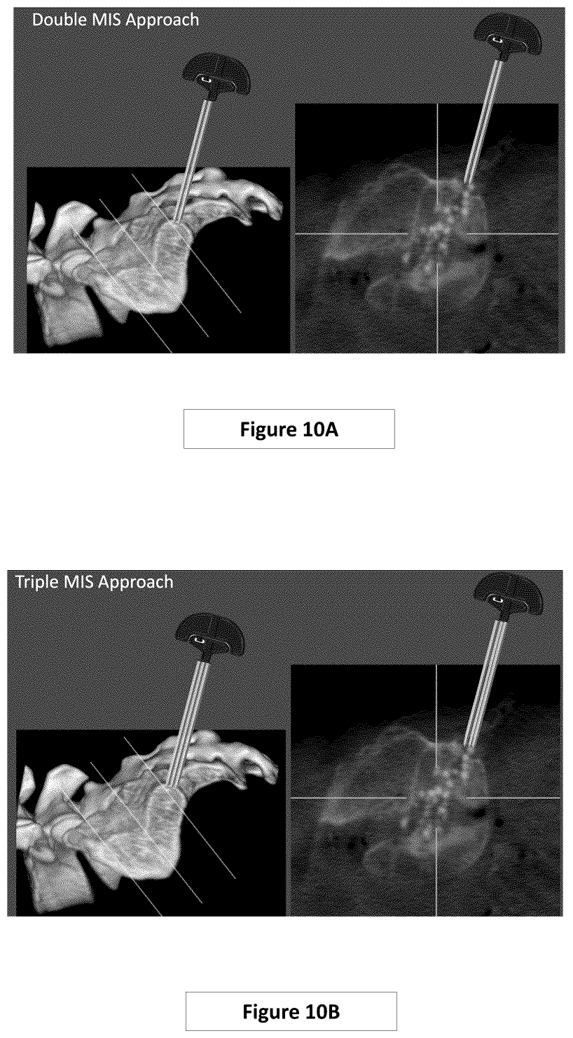

FIG. 10A illustrates using a dual cannula (i.e., a cannula with two access tubes) to deliver implants. FIG. 10B illustrates using a triple cannula (i.e., a cannula with three access tubes) to deliver implants. Use of cannulas with multiple access tubes can deliver an increased amount of implant over a shorter period of time to fill the joint. This is not possible with technologies based on inflexible implants that do not have the ability to adapt to the anatomy and fill the entirety of the joint to be fused. The amount of implants used can vary based on the patient's anatomy. This design allows for the proper nesting of multiple implants within the joint. This approach also allows various other surgical minimally disruptive approaches not available with current solutions. The described methods can insert the implants as a linear string (or individual beads) through a cannula, then the implant randomly folds in the joint to form a dense support with macro spaces for vascular through and inflow for enhanced osteointegration and subsequent bony fusion. This supports the anatomy by filling the SI joint, where the implant comprises osteoconductive material and/or osteoinductive material to provide support for bone growth and can become osteogenic combined with the patient's own blood and nutrients.

Although using cannulas with multiple tubes have been described as ways to deliver the implant, but multiple single tube cannulas can also be used. In addition, although described with respect to a posterior-caudal procedure, other anatomical approaches can also be used.

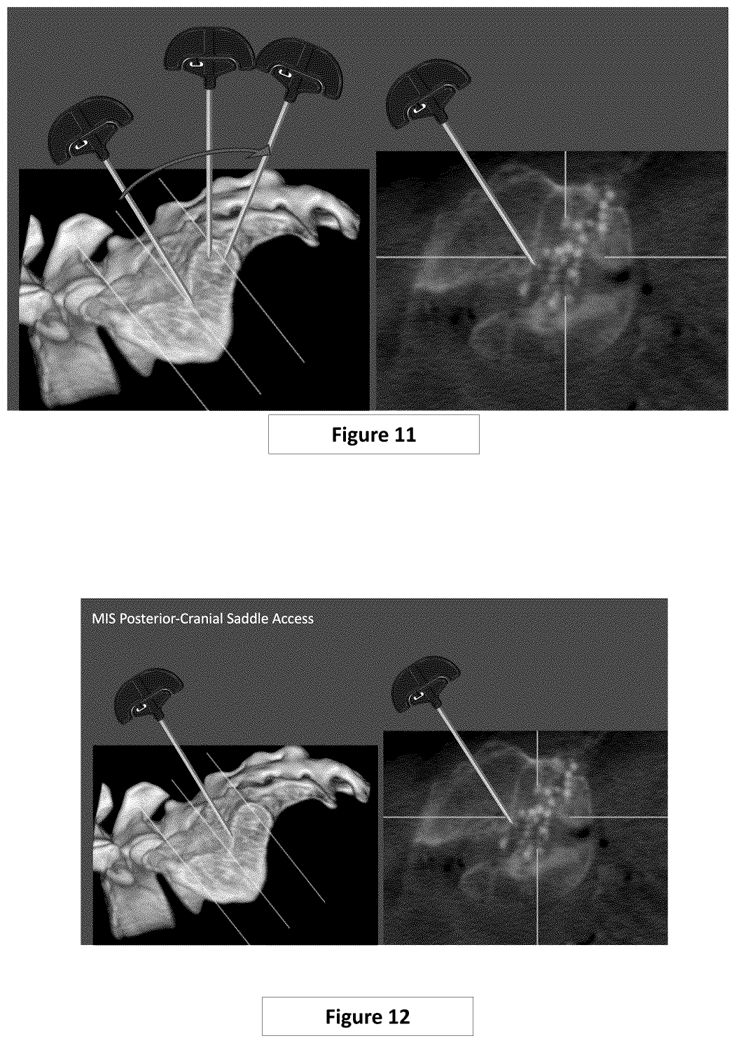

FIG. 11 illustrates the use of multiple single tube cannulas. The structural but flexibility of the implants enables performing the minimally invasive approach from several approach angles and entry points to the sacroiliac joint while avoiding harmful removal of bone or ligaments required by some systems. This is an important advantage to account for the complex anatomy of the SI joint. Due to the small size, even the dorsal-saddle approach to the joint is minimally invasive and the disruption to the dorsal interosseous ligament is minimized when compared to conventional systems.

FIG. 12 illustrates a posterior-cranial saddle access approach. In situations where a physician may find access blocking bony structures such as osteophytes in the area used for a posterior-caudal approach, the minimally invasive feature of this method will allow the physician to use the same general method and instrumentation to access the SI-Joint through the posterior saddle approach. Due to the minimally invasive feature, significant injury or even removal of vital anatomical structures is reduced or even entirely avoided. Despite this approach going through the intraosseous ligament, it provides minimal injury to the ligament as it will allow the splicing/spreading of the ligament versus significant cutting or removal.

The minimally invasive feature of this method and instrumentation allow swiveling or angulating the access cannula to direct the flexible implants to reach specific areas of the joint. The swiveling of the minimally invasive cannula in combination of the flexible implant allows the physician to steer and direct the implants to desired locations and assure maximum joint filling for best possible joint stability and fusion.

This anatomy sparing, minimally invasive method in combination with the flexible implant does not inhibit future, additional surgeries, in fact if additional surgeries are needed, only minimal, surgical tissue scaring may be present from the initial surgery, therefore significantly simplifying possible subsequent surgeries.

A number of embodiments of the systems and methods have been described. Nevertheless, it will be understood that various modifications may be made without departing from the spirit and scope of this specification. Accordingly, other embodiments are within the scope of the following claims.

What is claimed is:

1. A method for stabilizing a sacroiliac (SI) joint, the method comprising:
inserting a cannulated trocar/cannula assembly into the SI joint;
removing the cannulated trocar leaving a cannula extending into the SI joint;
inserting implants or a flexible chain of implants into the cannula;
inserting a pusher into cannula to advance the implants or chain of implants out of the cannula into an articular area of the SI joint.

2. The method of claim 1, further comprising placing a Kirschner wire posterior-caudal into the SI joint.

3. The method of claim 2, wherein inserting the cannulated trocar. The method of claim cannula assembly into the SI joint comprises inserting the cannulated trocar/cannula assembly into the SI joint along the Kirschner wire.

4. The method of claim 3, further comprising removing the k-wire.

5. The method of claim 1, further comprising rasping a surface of the SI joint to create bleeding bone.

6. The method of claim 1, wherein inserting the cannulated trocar/cannula assembly into the SI joint comprises inserting the cannulated trocar/cannula assembly into the SI joint at a location used for injection of steroids into the SI joint for pain relief.

7. The method of claim 1, wherein inserting a cannulated trocar/cannula assembly into the SI joint comprises using fluoroscopic-guided or CT guided insertion.

8. The method of claim 7, wherein the SI joint is localized using a fluoroscope to visualize an inferior component of the articular space.

9. The method of claim 7, further comprising performing a preliminary CT of the SI joint.

10. The method of claim 1, wherein inserting a cannulated trocar/cannula assembly into the SI joint in performed without damage or penetration to dorsal intraosseous ligaments.

11. The method of claim 1, further comprising identifying blocking structures in the area used for a posterior-caudal approach.

12. The method of claim 11, wherein, in response to identifying blocking structures in the area used for a posterior-caudal approach, inserting the cannulated trocar/cannula assembly into the SI joint comprises inserting the cannulated trocar/cannula assembly into the SI joint using a posterior saddle approach.

13. The method of claim 11, wherein the blocking structures comprise osteophytes.
